(19) United States
(12) Patent Application Publication
(10) Pub. No.: US 2024/0423542 A1
(43) Pub. Date: Dec. 26, 2024

(54) MEDICAL DEVICE

(71) Applicant: Nobilis Animo Limited, Sofia (BG)

(72) Inventors: Ivailo Zlatkov DACHOV, Sofia (BG); Racho Stefanov RIBAROV, Sofia (BG)

(73) Assignee: Nobilis Animo Limited, Sofia (BG)

(21) Appl. No.: 18/572,955

(22) PCT Filed: Oct. 12, 2023
(86) PCT No.: PCT/EP2023/025430

§ 371 Date: Dec. 21, 2023

(30) Foreign Application Priority Data
Dec. 19, 2022 (BG) BG/P/2022/113633
Jul. 11, 2023 (GB) GB2310610.7

(51) Int. Cl. A61B 5/00; A61B 5/021; A61B 5/024; A61B 5/1455

(52) U.S. Cl. CPC A61B 5/681; A61B 5/02116; A61B 5/02141; A61B 5/02427; A61B 5/02438; A61B 5/14552; A61B 5/7246; A61B 5/7271

(57) ABSTRACT

The present invention relates to physiological monitoring device and systems. In particular, the present invention is directed to a medical device configured to urge at least two physiological monitors with a nominal contact against skin of a wearer to measure and monitor physiological parameters. The medical device of the present invention can measure, evaluate or determine, continuously, in real-time, biomarkers or the like of a subject. More in particular, the present invention is directed to a medical device configured to continuously measure and monitor the pulse, SpO₂ or blood pressure of the wearer.

### BACKGROUND OF THE INVENTION

The present invention relates to physiological monitoring device and systems. In particular, the present invention is directed to a medical device configured to urge at least two physiological monitors with a nominal contact against skin of a wearer to measure and monitor physiological parameters. The medical device of the present invention can measure or evaluated, continuously, in real-time, biomarkers or the like of a subject. More in particular, the present invention is directed to a medical device configured to continuously measure and monitor the pulse, SpO₂ or blood pressure of the wearer.

Noninvasive, continuous and reliable physiological monitoring of for example pulse, SpO₂ and blood pressure, is vital to the healthcare of patients. Many noninvasive physiological monitoring systems, however, require that the device sensing the physiological parameters maintain an effective skin contact while at the same time relying on established approaches such as mathematical models, numerical simulations, empiric methods or comparative analysis, which deliver inconsistent and often inaccurate readings which can be misleading to physicians and patients alike. Moreover, most of the current noninvasive physiological monitoring devices and systems can be cumbersome e.g. ambulatory measurements of blood pressure using inflatable cuffs, and incapable of providing easy to use, continuous, accurate and reliable physiological parameters assessment which can be safely relied on.

As can be seen, there is an unmet need for a more accurate device configured to urge a physiological monitor with a nominal contact against the skin of the wearer for continuous, accurate and reliable physiological monitoring of for example pulse, SpO₂ and blood pressure. This unmet need is at least in part addressed by the present invention.

### SUMMARY OF THE INVENTION

In one aspect of the present invention there is provided a medical device comprising: a first monitoring interface having a main housing, the main housing terminating in opposing securing elements; a subsidiary module operatively associated with the main housing and a second monitoring interface; a distal end of the securing element incorporating a biasing component; wherein the biasing component is configured to urge the first monitoring interface against the skin of a wearer and urge a nominal contact force between the first monitoring interface and the dorsal wrist portion of the wearer, and wherein the biasing component is further configured to urge the second monitoring interface against the skin of a wearer and urge a nominal contact force between the second monitoring interface and the volar wrist portion of the wearer.

In some embodiments of the present invention there is provided a medical device comprising: a first monitoring interface having a main housing, the main housing terminating in opposing securing elements; a subsidiary module operatively associated with the main housing and a second monitoring interface; a distal end of the securing element incorporating a biasing component; wherein the biasing component is configured to urge the first monitoring interface against the skin of a wearer and urge a nominal contact force between the first monitoring interface and the dorsal wrist portion of the wearer, and wherein the biasing component is further configured to urge the second monitoring interface against the skin of a wearer and urge a nominal contact force between the second monitoring interface and the volar wrist portion of the wearer, and wherein operatively associated with the first monitoring interface and the second monitoring interface and contained in the main housing and the subsidiary module are two or more physiological monitor integers.

According to a further aspect the present invention provides a method for measuring the blood pressure in a subject using medical device comprising a first monitoring interface having a main housing, the main housing terminating in opposing securing elements; a subsidiary module operatively associated with the main housing and a second monitoring interface; a distal end of the securing element incorporating a biasing component; wherein the biasing component is configured to urge the first monitoring interface against the skin of a wearer and urge a nominal contact force between the first monitoring interface and the dorsal wrist portion of the wearer, and wherein the biasing component is further configured to urge the second monitoring interface against the skin of a wearer and urge a nominal contact force between the second monitoring interface and the volar wrist portion of the wearer, wherein the blood pressure is calculated using a formula based on a machine learning model that incorporates the correlation of the distance between inflection points in two blood pulse waves.

In some embodiments, the method of the present invention comprises measuring the blood pressure in a subject, wherein the blood pressure is calculated using a formula based on a machine learning model that incorporates the correlation of the distance between inflection points of the capillary pulse wave and the arterial pulse wave.

These and other features, aspects and advantages of the present invention will become better understood with reference to the following drawings, description, and claims.

### BRIEF DESCRIPTION OF THE DRAWINGS

FIG. 1. Medical device comprising first monitoring interface with sensor (3), connection point (4), a main housing (9) terminating in exemplary wrist straps comprising opposing securing elements (1), (2), (7) and (8) in opened configuration, and subsidiary module with second monitoring interface (6) and sensor (5). Connection (4) can be used as a charge point or a conduit between first monitoring interface (3) and subsidiary module with second monitoring interface (6).

FIG. 2. Medical device-comprising first monitoring interface having main housing with charging point (10).

FIG. 3. Schematic representation of medical device (a) readings or data collection (b), correlation between arterial blood pulse wave data and capillary blood pulse wave data, mathematical manipulation and software analysis of the data (c), transmittal and storage of the data on a Server/iCloud (d), and downstream uses of the collated physiological data (e).

FIG. 4. Medical device comprising first monitoring interface (3) having main housing which terminates in exemplary securing elements (wrist strap) in opened configuration and a separate but operatively associated with the first monitoring interface subsidiary module comprising a second monitoring interface with exemplary suction cup skin attachment securing elements. The connectivity between the first monitoring interface and the subsidiary module with the second monitoring interface may be via Bluetooth, WiFi or the like.

FIG. 5. Medical device comprising first monitoring interface having main housing terminating in opposing securing elements in closed configuration and subsidiary module. The connectivity between the first monitoring interface and the subsidiary module with the second monitoring interface may be via Bluetooth, WiFi or the like.

FIG. 6. A delay in pulse transit time (PTT) between both capillary blood pulse wave and arterial blood pulse wave has a direct link with changes in blood pressure.

### DETAILED DESCRIPTION OF THE INVENTION

Throughout this disclosure, various scientific publications, patents and published patent specifications are referenced by an identifying citation. The disclosures of these publications, patents and published patent specifications are hereby incorporated by reference into the present disclosure to more fully describe the state of the art to which this disclosure pertains.

As used herein, certain terms may have the following defined meanings.

As used in the specification and the claims, the singular form “a,” “an” and “the” include singular and plural references unless the context clearly dictates otherwise. For example, the term “biomarker” includes a single or plurality of biomarkers.

Throughout the specification, when a portion is “connected” or “coupled” to another portion, such as first monitoring interface to subsidiary module, this includes not only a case of being “directly connected or coupled” but also a case of being “electrically connected” with another element interposed therebetween, as well as for example “WiFi connected”, “Bluetooth connected” or the like.

Throughout the specification, when a member is said to be located “on” another member, this includes not only a case in which the member is in contact with another member but also a case in which there is another member between the two members.

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a medical device, comprising: a first monitoring interface having a main housing, the main housing terminating in opposing securing elements; a subsidiary module operatively associated with the first monitoring interface and a second monitoring interface; a distal end of the securing elements incorporating a biasing component; wherein the biasing component is configured to urge the first monitoring interface against the skin of a wearer and urge a nominal contact force between the first monitoring interface and the dorsal wrist portion of the wearer. Operatively associated with the first monitoring interface and the subsidiary module comprising a second monitoring interface, are two or more physiological monitor devices.

According to an aspect of the present invention, there is provided a medical device comprising: a first monitoring interface having a main housing, the main housing terminating in opposing securing elements; a subsidiary module operatively associated with the main housing and a second monitoring interface; a distal end of the securing element incorporating a biasing component; wherein the biasing component is configured to urge the first monitoring interface against the skin of a wearer and urge a nominal contact force between the first monitoring interface and the dorsal wrist portion of the wearer, and wherein the biasing component is further configured to urge the second monitoring interface against the skin of a wearer and urge a nominal contact force between the second monitoring interface and the volar wrist portion of the wearer.

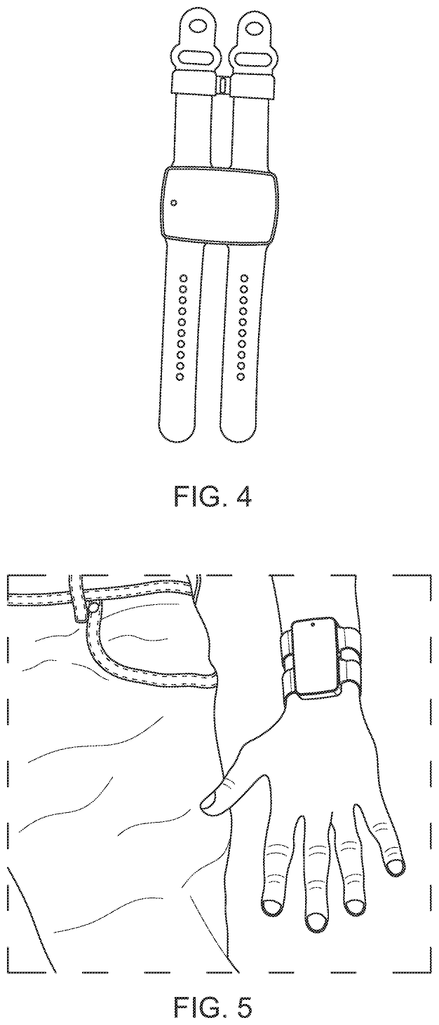

Referring to FIGS. 4 and 5, the present invention may include a medical device comprising first monitoring interface having main housing which terminates in opposing securing elements in opened or closed configuration and a separate but operatively associated with the first monitoring interface is a subsidiary module comprising a second monitoring interface.

In some embodiments of the present invention there is provided a medical device comprising: a first monitoring interface having a main housing, the main housing terminating in opposing securing elements; a subsidiary module operatively associated with the main housing and a second monitoring interface; a distal end of the securing element incorporating a biasing component; wherein the biasing component is configured to urge the first monitoring interface against the skin of a wearer and urge a nominal contact force between the first monitoring interface and the dorsal wrist portion of the wearer, and wherein the biasing component is further configured to urge the second monitoring interface against the skin of a wearer and urge a nominal contact force between the second monitoring interface and the volar wrist portion of the wearer, and wherein operatively associated with the first monitoring interface and the second monitoring interface and contained in the main housing and the subsidiary module are two or more physiological monitor integers.

In some embodiments of the present invention, the first monitoring interface and the subsidiary module with the second monitoring interface may be directly connected or via Bluetooth, WiFi or the like.

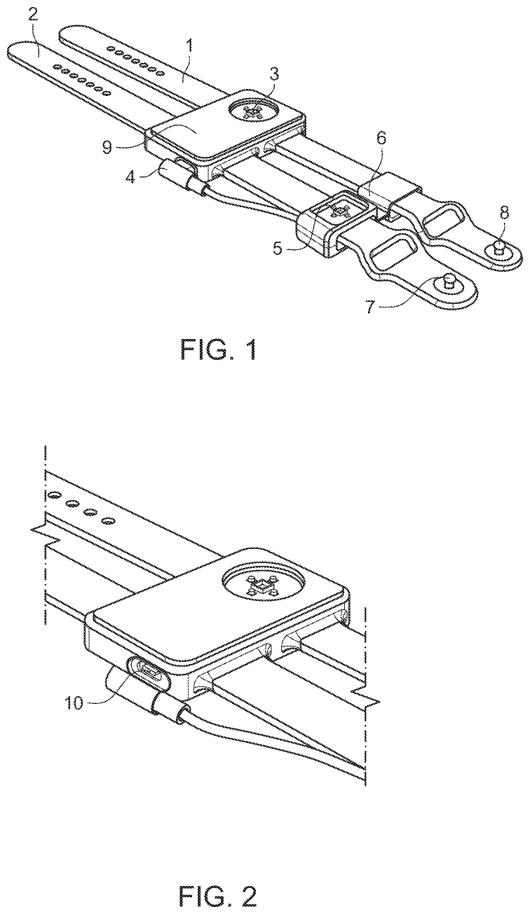

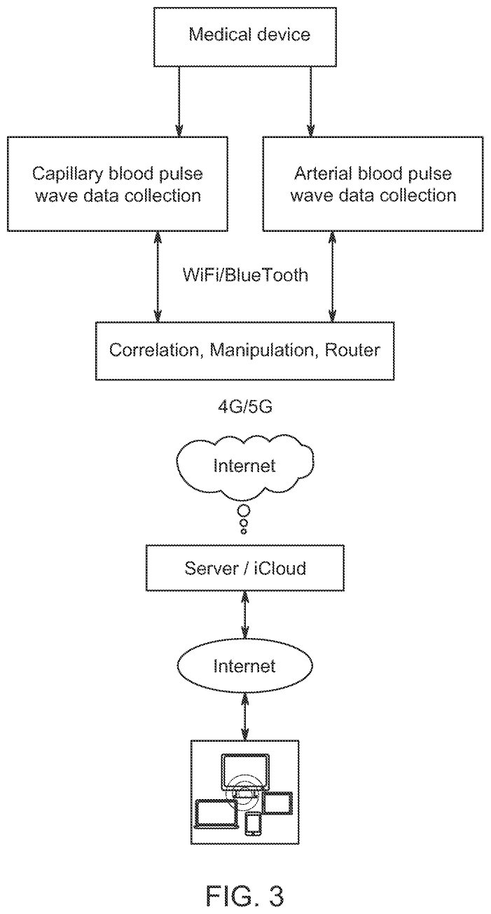

In some embodiments of the present invention, the first monitoring interface and the subsidiary module comprising a second monitoring interface can communicate and thus be operatively associated by direct connection. Referring here to FIG. 1 the present invention may include a medical device comprising first monitoring interface with sensor (3), connection point (4), a main housing (9) terminating in exemplary opposing securing elements (1), (2), (7) and (8) in opened configuration, and subsidiary module with second monitoring interface (6) and sensor (5). Connection (4) can be used as a charge point or a conduit between the first monitoring interface (3) and the subsidiary module with the second monitoring interface (6).

The two or more operatively associated physiological monitor integers may include sensors, biomarkers, or the like, that when the first monitoring interface and the second monitoring interface have effective skin contact, the two or more physiological monitor integers can sense or monitor physiological parameters.

The two or more operatively associated physiological monitor integers may include sensors, biomarkers, or the like, that when the first monitoring interface and the second monitoring interface have effective skin contact, the two or more physiological monitor integers can sense or monitor physiological parameters continuously.

The two or more operatively associated physiological monitor integers may include sensors, biomarkers, or the like, that when the first monitoring interface and the second monitoring interface have effective skin contact, the two or more physiological monitor integers can sense or monitor physiological parameters in real-time.

The two or more operatively associated physiological monitor integers may include sensors, biomarkers, or the like, that when the first monitoring interface and the second monitoring interface have effective skin contact, the two or more physiological monitor integers can sense or monitor predetermined physiological parameters.

The two or more operatively associated physiological monitor integers may include sensors, biomarkers, or the like, that when the first monitoring interface and the second monitoring interface have effective skin contact, the two or more physiological monitor integers can sense or monitor predetermined physiological parameters in real-time.

The two or more operatively associated physiological monitor integers may include sensors, biomarkers, or the like, that when the first monitoring interface and the second monitoring interface have effective skin contact, the two or more physiological monitor integers can sense or monitor predetermined physiological parameters continuously in real-time.

The two or more operatively associated physiological monitor integers may include sensors, biomarkers, or the like, that when the first monitoring interface and the second monitoring interfaces have effective skin contact, the two or more physiological monitor integers can sense, monitor or determine a disease or disorder.

The two or more physiological monitor integers may include sensors, biomarkers, or the like, that when the first monitoring interface and the second monitoring interface have effective skin contact, the two or more physiological monitor integers can sense, monitor or determine treatment of disease or disorder.

The two or more physiological monitor integers may include sensors, biomarkers, or the like, that when the first monitoring interface and the second monitoring interface have effective skin contact, the two or more physiological monitor integers can sense, monitor or determine treatment of disease or disorder continuously and/or in real-time.

The two or more physiological monitor integers may include sensors, biomarkers, or the like, that when the first monitoring interface and the second monitoring interface have effective skin contact, the two or more physiological monitor integers can sense, monitor or determine user activity, fitness, sleep and general wellness.

In some embodiments the securing element can be a strap. In some further embodiments the securing element can be a wrist strap.

In some embodiment, the biasing component may be a resilient material such as elastic, thermoplastic, rubber or spring.

In some embodiment, the biasing component can be one or more selected from the group consisting of pins and loops buckle, butterfly clasp, fold-over push-button deployment clasp, push-button deployment clasp, hidden deployment clasp, deployment clasp, Velcro strap, sliding buckle, friction-fit clickable clasp, resealable component, resealable magnetic component and resealable adhesive component.

Generally speaking, configuration of the securing elements and the biasing component are intended to hold the first monitoring interface and the second monitoring interface have effective skin contact.

As used in this application, the term “about” or “approximately” refers to a range of values within plus or minus 10% of the specified number. By way of non-limiting example, the term “about ten (10)” would encompass nine (9) to eleven (11) or 9-11. The term “substantially” refers to up to 80% or more of an entirety. Recitation of ranges of values herein are not intended to be limiting, referring instead individually to any and all values falling within the range, unless otherwise indicated, and each separate value within such a range is incorporated into the specification as if it were individually recited herein.

For purposes of this disclosure, the term “aligned” means parallel, substantially parallel, or forming an angle of less than 35.0 degrees. For purposes of this disclosure, the term “transverse” means perpendicular, substantially perpendicular, or forming an angle between 55.0 and 125.0 degrees. Also, for purposes of this disclosure, the term “length” means the longest dimension of an object. Also, for purposes of this disclosure, the term “width” means the dimension of an object from side to side. For the purposes of this disclosure, the term “above” generally means superjacent, substantially superjacent, or higher than another object although not directly overlying the object. Further, for purposes of this disclosure, the term “mechanical communication” generally refers to components being in direct physical contact with each other or being in indirect physical contact with each other where movement of one component affect the position of the other.

Generally speaking, subjects may be exposed to different monitoring equipment. The monitoring equipment may be for example general wellness/fitness type of monitoring equipment such as different smart watches or medical monitoring equipment such as ICU equipment. The boundaries between the general wellness/fitness monitoring equipment and the medical monitoring equipment is becoming more and more overlapping as a result of the ever, increasing understanding and appreciation of the physiologically relevance different bodily functions and biomarkers play in both wellness/fitness and the general health status of a subject.

The device of the present invention may be used to measure important bodily functions and parameters, such as for example heart rate, blood pressure, blood volume, oxygen levels, sugar levels, blood ketone levels, purine levels, liver enzyme activity and haemoglobin levels.

In some embodiments of the present invention, the device is configured to measure or monitor heart rate, blood pressure, blood volume, oxygen levels, sugar levels, blood ketone levels, purine levels, liver enzyme activity and haemoglobin levels on a subject.

In some embodiments of the present invention, the device is configured to measure or monitor continuously, heart rate, blood pressure, purine levels, sugar levels, blood ketone levels, liver enzyme activity and haemoglobin levels on a subject.

In some embodiments of the present invention, the device is configured to measure or monitor continuously in real-time, heart rate, blood pressure, purine levels, sugar levels, blood ketone levels, liver enzyme activity and haemoglobin levels on a subject.

Subjects can also be exposed to different types of monitoring equipment to measure and monitor for example biomarkers and/or parameters such as an electrocardiogram (ECG); respiration rate (RR); body mass index (BMI); alkaline phosphatase (ALP); alanine transaminase (ALT); aspartate aminotransferase (AST); arterial pH (Art pH); partial pressure of oxygen (PaO₂); oxygen saturation (SpO₂%); partial pressure of carbon dioxide (PaCO₂); red blood cell count (RBC); photoplethysmography (PPG); mean corpuscular haemoglobin concentration (MCHC); mean corpuscular haemoglobin (MCH); mean platelet volume (MPV); platelet distribution width (PDW); red cell distribution width (RDW); white blood cells (WBC); absolute neutrophil count (ANC); activated partial thromboplastin time (aPTT); partial thromboplastin time; hypoxanthine; pulse transit time (PTT), pulse; blood pressure; capillary pulse wave; arterial pulse wave and C-reactive protein (CRP).

Data collected by the medical device of the present invention can be processed and presented as mean standard deviation (mSD).

In some embodiments of the present invention, the medical device is configured to measure or monitor biomarkers and/or parameters such as an electrocardiogram (ECG); respiration rate (RR); body mass index (BMI); alkaline phosphatase (ALP); alanine transaminase (ALT); aspartate aminotransferase (AST); arterial pH (Art pH); partial pressure of oxygen (PaO₂); oxygen saturation (SpO₂%); partial pressure of carbon dioxide (PaCO₂); red blood cell count (RBC); mean corpuscular haemoglobin concentration (MCHC); mean corpuscular haemoglobin (MCH); photoplethysmography (PPG); mean platelet volume (MPV); platelet distribution width (PDW); red cell distribution width (RDW); white blood cells (WBC); absolute neutrophil count (ANC); activated partial thromboplastin time (aPTT); partial thromboplastin time; hypoxanthine; pulse transit time (PTT), pulse; blood pressure; capillary pulse wave; arterial pulse wave and C-reactive protein (CRP).

In some embodiments of the present invention, the medical device is configured to measure or monitor continuously biomarkers and parameters such as an electrocardiogram (ECG); respiration rate (RR); body mass index (BMI); alkaline phosphatase (ALP); alanine transaminase (ALT); aspartate aminotransferase (AST); arterial pH (Art pH); partial pressure of oxygen (PaO₂); oxygen saturation (SpO₂%); partial pressure of carbon dioxide (PaCO₂); red blood cell count (RBC); photoplethysmography (PPG); mean corpuscular haemoglobin concentration (MCHC); mean corpuscular haemoglobin (MCH); mean platelet volume (MPV); platelet distribution width (PDW); red cell distribution width (RDW); white blood cells (WBC); absolute neutrophil count (ANC); activated partial thromboplastin time (aPTT); partial thromboplastin time; hypoxanthine; pulse transit time (PTT), pulse; blood pressure; capillary pulse wave; arterial pulse wave and C-reactive protein (CRP).

In some embodiments of the present invention, the medical device is configured to measure or monitor continuously in real-time biomarkers and parameters such as an electrocardiogram (ECG); respiration rate (RR); body mass index (BMI); alkaline phosphatase (ALP); alanine transaminase (ALT); aspartate aminotransferase (AST); arterial pH (Art pH); partial pressure of oxygen (PaO₂); oxygen saturation (SpO₂%); partial pressure of carbon dioxide (PaCO₂); red blood cell count (RBC); mean corpuscular haemoglobin concentration (MCHC); mean corpuscular haemoglobin (MCH); mean platelet volume (MPV); platelet distribution width (PDW); red cell distribution width (RDW); white blood cells (WBC); absolute neutrophil count (ANC); activated partial thromboplastin time (aPTT); partial thromboplastin time; hypoxanthine; Pulse transit Time (PTT), pulse; blood pressure; capillary pulse wave; arterial pulse wave and C-reactive protein (CRP).

In some instances, sensing devices may be used for pulse oximetry, which may be an effective and quick way to monitor heart and lung function of a person. These pulse oximetry devices may be capable of evaluating the colour of blood as the amount of oxygen carried by the haemoglobin may affect the colour of blood. In some examples, a pulse oximetry device may be placed on a wearer to measure the oxygenation of the person's blood.

In some embodiments, the device of the present invention is configured to monitor, such as continuously monitor in real-time, partial pressure of oxygen PaO₂.

In some embodiments, the device of the present invention continuously monitors partial pressure of oxygen PaO₂.

In some embodiments, the device of the present invention monitors, such as continuously in real-time monitors, oxygen saturation SpO₂.

In some embodiments, the device of the present invention continuously monitors oxygen saturation SpO₂.

Monitoring, such as continuous monitoring or continuous monitoring in real-time of cardiac rhythm, is also contemplated in the present invention which can enable transformative diagnostic and patient management tools.

In some embodiments, sensors are incorporated in the device allowing continuous monitoring of cardiac rhythm.

In some embodiments, optical sensors are incorporated in the device allowing continuous monitoring of blood volume variations referred to as photoplethysmography (PPG) from which the heart rate and other physiological parameters can be extracted to inform about user activity, fitness, sleep, and general wellness.

In some embodiments, the device of the present invention monitors cardiac rhythm. In some embodiments, the device of the present invention monitors cardiac rhythm. In some embodiments, the device of the present invention continuously monitors cardiac rhythm.

In some embodiments, the medical device of the present invention monitors PPG. In some embodiments, the device of the present invention monitors PPG. In some embodiments, the device of the present invention continuously monitors PPG. In some embodiments, the device of the present invention continuously in real-time monitors PPG.

In some embodiments, sensors are incorporated in the medical device of the present invention allowing continuous monitoring of a subject's pulse. In some embodiments, sensors are incorporated in the medical device of the present invention allowing continuous monitoring of a subject's pulse rate.

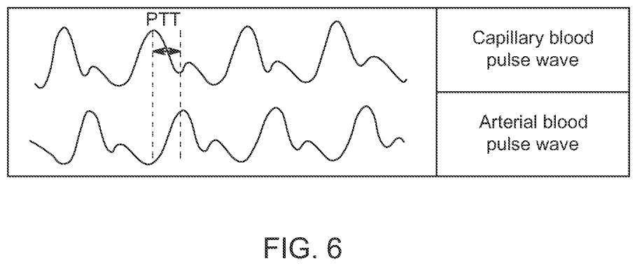

Referring here to FIG. 6 and without wishing to be bound by theory, a delay in pulse transit time (PTT) between the capillary blood pulse wave and arterial blood pulse wave has a direct link to changes in blood pressure. In other words, the ability to detect accurately and interpret meaningfully the delay observed at for example two separate points of reference on a subject between the capillary blood pulse wave and arterial blood pulse would be informative of the blood pressure of the subject.

In some embodiments, sensors are incorporated in the medical device of the present invention allowing continuous monitoring of a subject's PTT.

In some embodiments, the first monitoring interface and the second monitoring interface sense, monitor or determine the capillary pulse wave and the arterial pulse wave.

In some embodiments of the present invention, the capillary pulse wave and the arterial pulse wave are determined simultaneously, sequentially or concomitantly.

In some embodiments of the present invention, the capillary pulse wave and the arterial pulse wave are determined simultaneously.

In some embodiments of the present invention the simultaneously, sequentially or concomitantly determined capillary pulse wave and the arterial pulse wave are correlated.

In some embodiments of the present invention the simultaneously determined capillary pulse wave and the arterial pulse wave are correlated.

In some embodiments, the blood pressure is calculated using a formula based on a machine learning model that incorporates the correlation of the distance between inflection points in two pulse waves. In some embodiments, the blood pressure is calculated using a formula based on a machine learning model that incorporates the correlation of the distance between inflection points of the capillary pulse wave and the arterial pulse wave. A skilled person would be familiar with calculated using a formula based on a machine learning model that incorporates the correlation of the distance between inflection points is the different in the inflection points between the capillary pulse wave and the arterial pulse wave.

In some embodiments of the present invention, the capillary pulse wave and the arterial pulse wave are correlated using computational software analysis.

In some embodiments of the present invention, the capillary pulse wave and the arterial pulse wave are correlated using artificial intelligence (AI).

In some embodiments of the present invention, the capillary pulse wave and the arterial pulse wave are correlated using self-learning AI.

In some embodiments, sensors are incorporated in the medical device of the present invention allowing continuous monitoring of blood pressure. In some embodiments, sensors are incorporated in the medical device of the present invention allowing continuous monitoring of blood pressure in real-time.

### Physiological Monitor Devices

The skilled person in the art would appreciated that different physiological monitor integers can be incorporated in the medical device of the present invention.

It is contemplated that at least two physiological monitor integers can be incorporated in the medical device of the present invention.

The physiological monitor integers can be sensors, biomarkers, or the like and can be incorporated in the medical device to monitor or measure for example physiological parameters continuously or continuously in real-time on a subject.

In some embodiments, the device comprises an electrode sensor.

In some embodiments, the device comprises an optical sensor.

In some embodiments, the device comprises a light-emitting diode (LED). In some embodiments, the LED comprises a near-infrared LED.

In some embodiments, the device comprises a photodiode.

In some embodiments, the device comprises a laser.

In an embodiment, the device comprises two or more from the group consisting or an electrode sensor, an optical sensor, light-emitting diode (LED) sensor, a near-infrared LED sensor, a photodiode sensor and a laser.

In some embodiments, the main housing and the subsidiary module may each include a strain gauge-based semiconductor thin film sensor, a capacitive thin film sensor for detecting capacity change according to a pressure, a piezoresistive sensor using a piezo resistance effect, or other various pressure sensors.

Without wishing to be bound by theory, the choice of physiological monitor device may be influenced by different factors such as for instance the type of physiological biomarker or parameter which is to be monitored or measures. Other contributory factors to the choice of physiological monitor device include proximity of the blood vessels to the skin surface around the first monitoring interface and the subsidiary module. The accurate measurement of blood pressure is essential for example the diagnosis and management of hypertension. Human error is also a factor as well as the place where measurements are taken for example in an office setting or out and about setting with multiple measurement being taken.

A particular factor which may influence the choice of the physiological monitor device, especially when assessing or determining for example blood pressure is the proximity of the device to the heart of the wearer. The proximity to the heart may facilitate more accurate measurements when detecting or monitoring physiological biomarkers or parameters such as for instance heart rate, pulse, blood oxygen level, blood pressure, capillary pulse wave or arterial pulse wave.

A further factor which may influence the choice of the physiological monitor include the distance between the first monitoring interface and the subsidiary module. In order to increase accuracy of the measurements the subsidiary module is configured to slide or move along the length of the biasing component. The distance or juxtaposition of the subsidiary module relative to the first monitoring interface may thus freely be modified or adjusted by the wearer to achieve the desired nominal contact with the volar and/or dorsal portion of the wrist.

In some embodiments of the present invention the subsidiary module is configured to slide or move along the length of the biasing component.

Current blood pressure (BP) measurement devices employ an inflatable cuff and thus cannot be used anytime or anywhere to manage for example hypertension. Pulse transit time (PTT) varies inversely with BP in a person due to the physical properties of arteries and can be obtained without a cuff. As a result, PTT is being widely pursued for cuff-less BP measurement. The present invention provides a medical device which is capable of cuff-less BP measurement which is easy to use and accurate.

Additionally, the physiological monitor medical device of the present invention may be useful for controlled environments. The device may be particularly useful for monitoring daily routine activities, during fitness training or during sleep.

In particular, the physiological monitor medical device would allow richer data source to be accessed, leading to more accurate monitoring of physiological biomarkers or parameters and confidence in the data especially blood pressure monitoring.

The present inventors have surprisingly observed that the medical device of the present invention provides an easy to use, consistently accurate, continuous and reliable blood pressure determination when there is achieved both a nominal contact force between the first monitoring interface and the dorsal wrist portion of the wearer and a nominal contact force between the second monitoring interface and the volar wrist portion of the wearer.

According to a further aspect the present invention provides a method for measuring the blood pressure in a subject using medical device comprising a first monitoring interface having a main housing, the main housing terminating in opposing securing elements; a subsidiary module operatively associated with the main housing and a second monitoring interface; a distal end of the securing element incorporating a biasing component; wherein the biasing component is configured to urge the first monitoring interface against the skin of a wearer and urge a nominal contact force between the first monitoring interface and the dorsal wrist portion of the wearer, and wherein the biasing component is further configured to urge the second monitoring interface against the skin of a wearer and urge a nominal contact force between the second monitoring interface and the volar wrist portion of the wearer, wherein the blood pressure is calculated using a formula based on a machine learning model that incorporates the correlation of the distance between inflection points in two blood pulse waves.

In some embodiments, the method of the present invention comprises measuring the blood pressure in a subject, wherein the blood pressure is calculated using a formula based on a machine learning model that incorporates the correlation of the distance between inflection points of the capillary pulse wave and the arterial pulse wave.

### Data Processing

In some embodiments, the data collected from the device has to be processed on the device or remotely. After normalisation and pre-processing of the data, MetIDQ™ software (Biocrates) can be used for peak integration and calculation of biomarkers. If the measurements were outside the measurable range, values were imputed as follows: concentrations below the detection limit (LOD) was set to half of the lowest measured concentrations. Concentrations below the limit of quantification (LOQ) can be set to half of the LOQ. In addition, concentration higher than the highest calibration standard concentration can be set to the highest standard concentrations. In some further embodiments, the data collected from the device has to be processed using machine learning and simulated training data.

The present invention recognises that personal information data, including the physiological biomarker data acquired using the physiological monitor device of the present invention, can be used for the benefit of the wearer and/or others.

Further, other uses for personal information data, including biometric data that benefit the user are also contemplated by the present disclosure.

The present disclosure further contemplates that the entities that may be responsible for the collection, analysis, disclosure, transfer, storage, or other use of any personal information data will comply with well-established privacy policies and/or privacy practices. In particular, such entities should implement and consistently use privacy policies and practices that are generally recognised as meeting or exceeding industry or governmental requirements for maintaining personal information data private and secure, including the use of data encryption and security methods that at least meets or may even exceed industry or government standards.

An embodiment of the present disclosure may also be implemented in the form of a recording medium including instructions executable by a computer, such as a program module executed by the computer. Computer-readable media may be any available media that may be accessed by a computer and include both volatile and nonvolatile media and removable and non-removable media.

In addition, the computer-readable media may include all computer storage media. The computer storage media includes both volatile and nonvolatile media and removable and non-removable media implemented by any method or technology of storing information, such as a computer readable instruction, a data structure, a program module, and other data.

In some embodiments, the collected data is calculated using a formula based on a machine learning model that incorporates the correlation of the distance between inflection points in two pulse waves. In some embodiments, the collected data is calculated using a formula based on a machine learning model that incorporates the correlation of the distance between inflection points of the capillary pulse wave and the arterial pulse wave.

### Statistical Analysis

Analysis can be carried out on the device itself or remotely using systems and software programs known in the art such as for example IBM SPSS version 25. Variables in measurement and data with skewed distributions can be log-transformed to ensure normality. Comparisons can be performed with t-test, Wilcoxon-Mann-Whitney, and one-way ANOVA as appropriate. Significance was defined as p<0.05. Non-parametric tests were used for comparing ordinal or non-normal variables. Data can be presented as mean standard deviation (mSD).

### Definitions

As used herein, the term “biomarker” refers to a physiological characteristic or physiological parameter that can be objectively measured by for example known sensors and evaluated for example by using known statistical methods, as an indicator of normal and/or disease processes, pharmacological responses or physiological status of a subject. A “biomarker” can be used to measure the onset or the progress of a disease, the effects of treatment or regimen, or provide information on user activity, fitness, sleep, health and metabolic status. One of the advantages of the medical device described herein is that biomarker measurements can be collected without disruption of the skin or direct contact with the blood supply of the wearer.

In some embodiment, the biomarker is a predetermined physiological parameter.

In some embodiments, the parameter can be chemical, physical or biological.

The parameter can be altered or modified where the alteration can be measured or evaluated continuously or continuously in real-time by the device in situ i.e. while in place on a subject.

The parameter can be altered or modified where the alteration can be measured or evaluated continuously or continuously in real-time by the device remotely.

In some embodiments, the biomarker or parameter can be selected from one or more of the group consisting of electrocardiogram (ECG); respiration rate (RR); cardiac rhythm (CR), photoplethysmography (PPG); body mass index (BMI); alkaline phosphatase (ALP); alanine transaminase (ALT); aspartate aminotransferase (AST); arterial pH (Art pH); partial pressure of oxygen (PaO₂); oxygen saturation (SpO₂%); partial pressure of carbon dioxide (PaCO₂); red blood cell count (RBC); mean corpuscular haemoglobin concentration (MCHC); mean corpuscular haemoglobin (MCH); mean platelet volume (MPV); platelet distribution width (PDW); red cell distribution width (RDW); white blood cells (WBC); absolute neutrophil count (ANC); activated partial thromboplastin time (aPTT); partial thromboplastin time (PTT); hypoxanthine; pulse transit time (PtT), pulse; blood pressure; capillary pulse wave; arterial pulse wave and C-reactive protein (CRP).

As used herein, the term “alteration” may be used interchangeably with the terms, “alter” or “modify” such as increase or decrease in the level of a metabolite such as a chemical or a biomarker detected and/or analysed and/or monitored, by the device of the present invention.

In some embodiments, the alteration or delay is at least 0.001%, 0.005%, 0.01%, 0.05%, 0.08%, 0.1%, 0.15%, 0.2%, 0.4%, 1%, 2%, 2.5%, 5%, 10%, 15%, 20%, 25%, 30%, 35%, 40%, 45%, 50%, 55%, 60%, 65%, 70%, 75%, 80%, 85%, 90%, 95% or 100% or greater compared to control or base level.

In some embodiments the alteration or delay may be at least 0.1, 0.25, 0.5, 1, 1.5, 2, 2.5, 3, 3.5, 4, 4.5, 5, 5.5, 6, 6.5, 7, 7.5, 8, 8.5, 9, 9.5, or 10-fold or greater compared to control or base level.

In some embodiments the alteration of the parameter is statistically significant. In some embodiments the effectiveness or the device of the present invention is determined qualitatively.

In some embodiments the effectiveness of the device of the present invention is determined quantitatively.

In some embodiments the alteration is determined qualitatively. In some embodiments the alteration is determined quantitatively.

In some embodiments, alteration is assessed by a qualitative step and/or a quantifying step. In further embodiments, the qualitative step and/or a quantifying step is performed on a sample using the device of the present invention.

In further embodiments, the qualitative step and/or a quantifying step is performed on a subject using the device of the present invention.

In further embodiments, the qualitative step and/or a quantifying step is performed continuously.

In further embodiments, the qualitative step and/or a quantifying step is performed in real-time. In further embodiments, the qualitative step and/or a quantifying step is performed continuously in real-time.

In further embodiments, the present disclosure the capillary pulse wave and the arterial pulse wave are determined simultaneously, sequentially or concomitantly.

In further embodiments, the present disclosure may also be implemented in the form of a recording medium including instructions executable by a computer, such as a program module executed by the computer. Computer-readable media may be any available media that may be accessed by a computer and include both volatile and nonvolatile media and removable and non-removable media. In addition, the computer-readable media may include all computer storage media. The computer storage media includes both volatile and nonvolatile media and removable and non-removable media implemented by any method or technology of storing information, such as a computer readable instruction, a data structure, a program module, and other data. The storage may be in the iCloud.

Although the device and system according to the present disclosure are described with reference to specific embodiments, some or all of their components or operations may be implemented by using a computer system having a general-purpose hardware architecture.

As used herein, “treating” or “treatment” of any disease or disorder refers, in certain embodiments, to ameliorating a disease or disorder that exists in a subject. “Treating” or “treatment” includes ameliorating at least one physiological or physical parameter, which may be indiscernible by the subject. In yet another embodiment, “treating” or “treatment” includes modulating the disease or disorder, either physically (e.g., stabilization of a discernible symptom) or physiologically (e.g., stabilisation of a physical parameter) or both.

In yet another embodiment, “treating” or “treatment” includes delaying or preventing the onset of the disease or disorder.

Further, as used herein, “treatment” includes preventing or delaying the recurrence of the disease, delaying or slowing the progression of the disease, ameliorating the disease state, providing a remission (partial or total) of the disease, decreasing the dose of one or more other medications required to treat the disease, delaying the progression of the disease, increasing or improving the quality of life, increasing weight gain, and/or prolonging survival. Also encompassed by “treatment” is a reduction of pathological consequence of a disease e.g., metabolic condition, disorder or severe illness.

As used herein the terms “subject” and “wearer” are used interchangeably. As used herein, the term “subject” means any animal, such as a vertebrate, preferably a mammal such as human. Preferably the subject wears or has been fitted with the medical device of the present invention.

The use of any and all examples, or exemplary language (“e.g.,” “such as,” or the like) provided herein, is intended merely to better illuminate the embodiments and does not pose a limitation on the scope of the embodiments or the claims. No language in the specification should be construed as indicating any unclaimed element as essential to the practice of the disclosed embodiments.

In the following description, it is understood that terms such as “first,” “second,” “top,” “bottom,” “up,” “down”, “upper”, “lower”, “above”, “below”, “beneath”, “front”, “back”, “over”, “under”, “left”, “right”, “dorsal”, “volar”, “palm side”, “back side” etc. are used with reference to the orientation of some of the components of the medical device of the present invention. Since constituents or components in various embodiments described here can be positioned in a number of different orientations, locations of the body, directional terminology is used for purposes of illustration only and is in no way limiting. The directional terminology is intended to be construed broadly, and therefore should not be interpreted to preclude components being oriented in different ways.

The disclosure illustratively described herein can suitably be practiced in the absence of any element or elements, limitation or limitations, not specifically disclosed herein. Thus, for example, the terms “comprising”, “including,” containing”, etc. shall be read expansively and without limitation.

Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure claimed. It will also be appreciated that the device(s), method(s), use(s), detector(s), sensor(s), physiological biomarker(s) may be subject to numerous rearrangements, modifications and substitutions without departing from the scope of the present disclosure as set forth and defined by the following claims.

1. A medical device, comprising:
a first monitoring interface having a main housing, the main housing terminating in opposing securing elements;
a subsidiary module operatively associated with the first monitoring interface and a second monitoring interface;
a distal end of the securing elements incorporating a biasing component;
wherein the biasing component is configured to urge the first monitoring interface against the skin of a wearer and urge a nominal contact force between the first monitoring interface and the dorsal wrist portion of the wearer; and
wherein the biasing component is further configured to urge the subsidiary module against the skin of a wearer and urge a nominal contact force between the second monitoring interface and the volar wrist portion of the wearer.

2. A medical device according to claim 1, wherein operatively associated with the first monitoring interface and the second monitoring interface, and contained in the main housing and the subsidiary module are two or more physiological monitor integers.

3. A medical device according to claim 2, wherein the two or more operatively associated physiological monitor integers include sensors, biomarkers, or the like, that when the first monitoring interface and the second monitoring interface have effective skin contact, the two or more physiological monitor devices sense or monitor physiological parameters.

4. A medical device according to claim 2, wherein the two or more operatively associated physiological monitor integers include sensors, biomarkers, or the like, that when the first monitoring interface and the second monitoring interfaces have effective skin contact, the two or more physiological monitor devices sense, monitor or determine a disease or disorder.

5. A medical device according to claim 4, wherein the two or more operatively associated physiological monitor devices integers sensors, biomarkers, or the like, that when the first monitoring interface and the second monitoring interfaces have effective skin contact, the two or more physiological monitor devices sense, monitor or determine treatment of disease or disorder.

6. A medical device according to claim 2, wherein the two or more operatively associated physiological monitor integers include sensors, biomarkers, or the like, that when the first monitoring interface and the second monitoring interfaces have effective skin contact, the two or more physiological monitor devices sense, monitor or determine treatment of disease or disorder continuously and/or in real-time.

7. A medical device according to claim 2, wherein the two or more operatively associated physiological monitor integers include sensors, biomarkers, or the like, that when the first monitoring interface and the second monitoring interface have effective skin contact, the two or more physiological monitor devices sense, monitor or determine user activity, fitness, sleep and general wellness.

8. A medical device according to claim 3, wherein the biomarker is one or more of the group consisting of electrocardiogram (ECG); respiration rate (RR); cardiac rhythm (CR), photoplethysmography (PPG); body mass index (BMI); alkaline phosphatase (ALP); alanine transaminase (ALT); aspartate aminotransferase (AST); arterial pH (Art pH); partial pressure of oxygen (PaO2); oxygen saturation (SpO2%); partial pressure of carbon dioxide (PaCO₂); red blood cell count (RBC); mean corpuscular haemoglobin concentration (MCHC); mean corpuscular haemoglobin (MCH); mean platelet volume (MPV); platelet distribution width (PDW); red cell distribution width (RDW); white blood cells (WBC); absolute neutrophil count (ANC); activated partial thromboplastin time (aPTT); partial thromboplastin time; hypoxanthine; pulse transit time (PTT); pulse; blood pressure; capillary pulse wave; arterial pulse wave and C-reactive protein (CRP).

9. A medical device according to claim 3, wherein the biomarker is partial pressure of oxygen PaO2.

10. A medical device according to claim 3, wherein the biomarker is oxygen saturation SpO2.

11. A medical device according to claim 3, wherein the biomarker is cardiac rhythm.

12. A medical device according to claim 3, wherein the biomarker is pulse transit time (PTT).

13. A medical device according to claim 3, wherein the biomarker is pulse.

14. A medical device according to claim 3, wherein the biomarker is blood pressure.

15. A medical device according to claim 8, wherein the first monitoring interface and the second monitoring interface sense, monitor or determine the capillary pulse wave and the arterial pulse wave.

16. A medical device according to claim 15, wherein the capillary pulse wave and the arterial pulse wave are determined simultaneously, sequentially or concomitantly.

17. A medical device according to claim 15, wherein the capillary pulse wave and the arterial pulse wave are determined simultaneously.

18. A medical device according to claim 16, wherein the capillary pulse wave and the arterial pulse wave are correlated.

19. A medical device according to claim 16, wherein the capillary pulse wave and the arterial pulse wave are calculated using a formula based on a machine learning model that incorporates the correlation of the distance between inflection points of the capillary pulse wave and the arterial pulse wave.

20. A medical device according to claim 19, wherein the capillary pulse wave and the arterial pulse wave are correlated using computational software analysis.

21. A medical device according to claim 1, wherein a subject is monitored continuously.

22. A medical device according to claim 1, wherein a subject is monitored continuously in real-time.

23. A medical device according to claim 1, wherein the sensor is selected from one or more of the group consisting of electrode sensor, optical sensor, light-emitting diode (LED), a near-infrared LED, photodiode, and a laser.

24. A medical device according to claim 1, wherein the sensor is an optical sensor.

25. A medical device according to claim 1, wherein the biasing component can be one or more selected from the group consisting of pins and loops buckle, butterfly clasp, fold-over push-button deployment clasp, push-button deployment clasp, hidden deployment clasp, deployment clasp, Velcro strap, sliding buckle, friction-fit clickable clasp, resealable component, resealable magnetic component and resealable adhesive component.

26. A method of measuring the blood pressure in subject using the devise according to claim 1, wherein the blood pressure is calculated using a formula based on a machine learning model that incorporates the correlation of the distance between inflection points in two blood pulse waves.

27. A method according to claim 26, wherein the blood pressure is calculated using a formula based on a machine learning model that incorporates the correlation of the distance between inflection points of the capillary pulse wave and the arterial pulse wave.
